(19) United States
(12) Patent Application Publication
(10) Pub. No.: US 2011/0281368 A1
(43) Pub. Date: Nov. 17, 2011

(54) NANOPARTICLE DERIVATIZATION OF TARGETS FOR DETECTING AND DETERMINING THE CONCENTRATIONS OF TARGETS BY IMPEADANCE-SPECTROSCOPY-BASED SENSORS

(75) Inventors: Andrei L. Gindilis, Vancouver, WA (US); Kevin Robert Schwarzkopf, Camas, WA (US); Paul J. Schuele, Washougal, WA (US); Mark Albert Crowder, Portland, OR (US)

(73) Assignee: Sharp Laboratories of America, Inc.

(21) Appl. No.: 12/780,270

(22) Filed: May 14, 2010

(51) Int. Cl. G01N 33/48; G01N 27/00

(52) U.S. Cl. 436/94; 422/82.02; 977/773; 977/762

(57) ABSTRACT

Embodiments of the present invention are directed to for detecting the presence and concentration of one or more particular target molecules in solutions, air or other gasses, or otherwise present in an environment or sample, by impedance-spectroscopy-based sensors. Various embodiments of the present invention provide for derivatizing target molecules with nanoparticles to increase capacitance changes at electrode surfaces in order to generate stronger signals and improve signal-to-noise ratios of impedance-spectroscopy-based sensors.

### TECHNICAL FIELD

The present invention is related to sensors that generate electromagnetic signals when bound to targets and, in particular, to efficient and reliable sensors that produce large signal-to-noise ratios.

### BACKGROUND OF THE INVENTION

Enormous research and development efforts have been made, during the past 100 years, to develop sensors that detect the presence of target molecules, target particles, or other target objects in solutions, air or other gasses, adsorbed to surfaces, or otherwise present in an environment or sample. With the advent of modern microelectronic, sub-microelectronic, microelectromechanical, and sub-microelectromechanical fabrication technologies, a wide variety of different types of sensors have been developed for commercial use. Sensors may be macroscale devices that include arrays of microscale sensor elements, such as oligonucleotide-probe-based microarrays, or may be microscale, sub-microscale, or nanoscale electromechanical, electro-optical, or optical-mechanical subcomponents of microelectromechanical devices, and microfluidic devices. A wide variety of different types of sensors are used in analytical instruments, diagnostics, and scientific instrumentation. As with many other types of technology, sensors are often characterized by various parameters of importance to researchers, designers, and manufacturers of sensor-based devices and equipment, including cost, sensitivity, specificity, viability, reusability, durability, and flexibility in application. Researchers, designers, and manufacturers of sensors and sensor-based devices and equipment continue to seek new sensor technologies that provide low-cost, reliable, durable, reusable, sensitive, and highly specific sensors that can be as broadly applied as possible to a variety of problem domains.

### SUMMARY OF THE INVENTION

Embodiments of the present invention are directed to for detecting the presence and concentration of one or more particular target molecules in solutions, air or other gasses, or otherwise present in an environment or sample, by impedancc-spectroscopy-based sensors. Various embodiments of the present invention provide for derivatizing target molecules with nanoparticles to increase capacitance changes at electrode surfaces in order to generate stronger signals and improve signal-to-noise ratios of impedance-spectroscopy-based sensors.

### BRIEF DESCRIPTION OF THE DRAWINGS

FIG. 1 illustrates a target-sensing problem domain that provides a context for a description of certain embodiments of the present invention.

FIG. 2 illustrates a sensor-based determination of target concentration within the exemplary problem domain illustrated in FIG. 1.

FIG. 3 illustrates an impedance-spectroscopy-based sensor.

FIGS. 4A-B illustrate the relative phase changes of current and voltage waveforms in an AG electrical circuit induced by a capacitor and inductor, respectively.

FIG. 5 illustrates a simple form of the impedance-spectroscopy-based sensor discussed above with reference to FIG. 3.

FIG. 6 shows the equivalent circuit for the sensor shown in FIG. 5.

FIG. 7 illustrates the sensor shown in FIG. 5 with a bound target biopolymer.

FIGS. 8A-B illustrate impedance-versus-time curves generated during introduction of a biopolymer target into the solution in which an impedance-spectroscopy-based sensor is immersed.

FIG. 9 illustrates a short oligonucleotide.

FIGS. 10A-B illustrate the hydrogen bonding between the purine and pyrimidine bases of two anti-parallel DNA strands.

FIG. 11 illustrates a short section of a DNA double helix comprising a first strand and a second, anti-parallel strand.

FIG. 12 illustrates an approach generally used for binding target molecules to impedance-spectroscopy-sensor electrodes.

FIG. 13 illustrates the approach of certain embodiments of the present invention to enhance the signal strength and signal-to-noise ratio of impedance-spectroscopy-based sensors.

FIGS. 14-15 illustrate a target-amplification method for generating a nanoparticle/target complex according to one embodiment of the present invention.

FIG. 16 illustrates an alternative approach to producing nanoparticle-bound target complexes.

FIGS. 17A-B illustrate an additional feature of nanoparticle/target complexes useful in improving signal-to-noise ratios of impedance signals generated by impedance-spectroscopy-based sensors according to certain embodiments of the present invention.

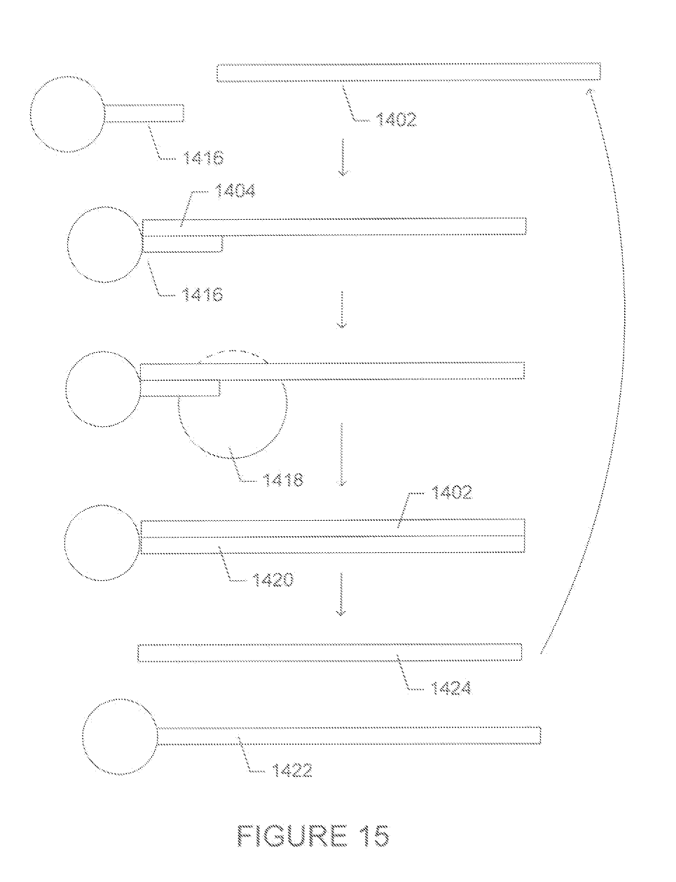

FIG. 15 shows the impedance response of ah ADK functionalized chip to ADK functionalized beads (specific response) and to P2P5 functionalized beads (negative control).

FIG. 19 shows the impedance response of P2P5 functionalized chip to P2P5-functionalized beads (specific response) and to ADK-functionalized beads (negative control).

FIG. 20 shows that addition of a nanowire suspension results in a rapid decrease in impedance of an impedance-spectroscopy-based sensor due to sedimentation of the conductive rods over the chip surface.

### DETAILED DESCRIPTION OF THE INVENTION

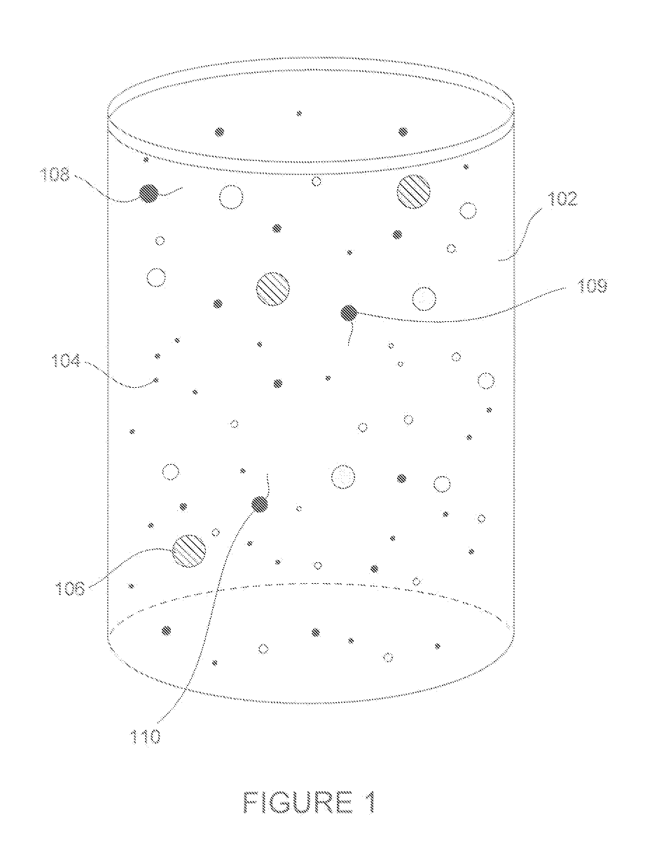

FIG. 1 illustrates a target-sensing problem domain that provides a context for a description of certain embodiments of the present invention. FIG. 1 shows a small, enclosed volume 102 of a complex solution containing symbolic representations of various different small molecules and ions, such as small molecule 104, various non-target biopolymers, such as non-target biopolymer 106, as well as a target biopolymer, three instances of which 108-110 are shown in the small enclosed volume 102 illustrated in FIG. 1. FIG. 1 is not intended to provide an accurate, scale-correct rendering of an enclosed volume of a complex solution, but is instead intended to illustrate the complexity of the solution 102 as well as to introduce symbolic illustration conventions for sensor targets and for non-target entities within the solution. Considering the enclosed volume of solution to be a sample, one common problem domain constitutes determining the concentration of target entities 108-110. [t], in the small volume 102 of the solution illustrated in FIG. 1.

There are many highly accurate, mature, and generally complex technologies for determining concentrations of targets in solutions and other media. These methods include a wide variety of different types of chromatography techniques, gel electrophoresis, analytical centrifugation, fluorescent-antibody assays, and many other methods. In general, any particular method, particularly the classical biochemical methods, can be used only for a subset of the sensing problem domains. For example, many methods require a minimum volume of solution for analysis. In addition, methods generally can detect targets reliably only over particular concentration ranges. Targets may often interact with each other and/or with other molecules, panicles, or other entities in the solution in ways that interfere with accurate determination of target concentration. Many methods require particular solvents, and cannot be used for other solvents. Many methods are time-consuming, expensive, and require complex instrumentation and laboratory equipment, and cannot therefore be carried out in real time or under field conditions.

For all of the above reasons, enormous effort has been undertaken, in recent years, to develop and commercialize highly specific, inexpensive, small, reliable, and sensitive sensors for detecting a wide variety of different targets in solutions, in air, adsorbed to different surfaces, substrates, and entities, and in other media and environments. The sensors may be used for environmental monitoring, biowarfare-agent detection, explosives detection, analysis of biopolymer and small-molecule solutions, detection of impurities in manufacturing quality control, and for a wide variety of additional applications, including diagnostics and scientific-research applications.

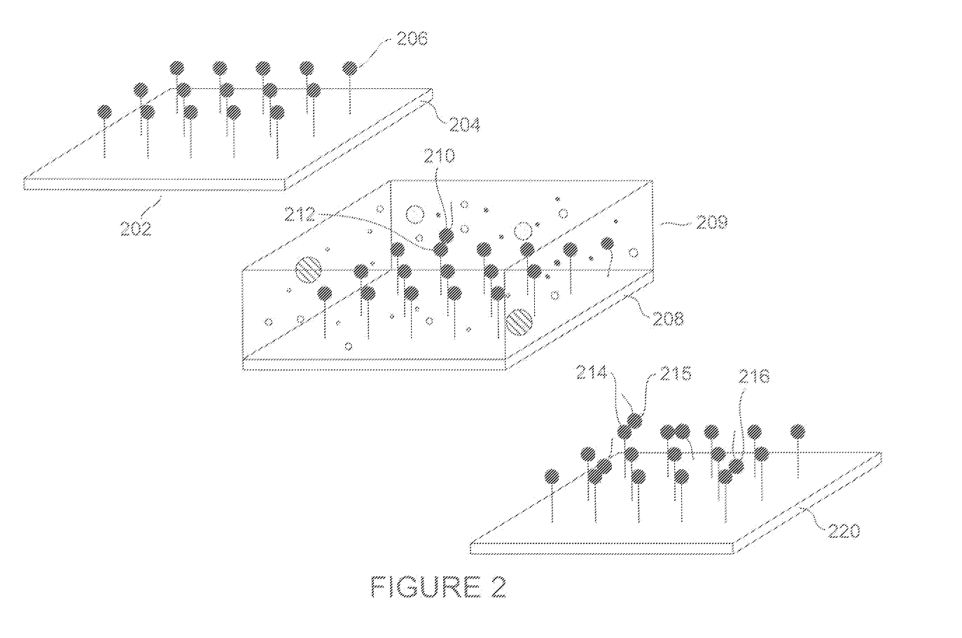

In general, a sensor is a signal transducer that responds to a target concentration or target presence, in a defined environment, by generating an electromagnetic signal, including electrical current and voltage signals, optical signals, radio-frequency signals, and other types of signals that can be detected and quantitatively evaluated by electronic devices, generally microelectronic, microprocessor-controlled devices. FIG. 2 illustrates a sensor-based determination of target concentration within the exemplary problem domain illustrated in FIG. 1. In FIG. 2, a small portion 202 of a sensor is shown to include a substrate 204 and target-binding probes, such as probe 206. Different types of sensors may include different numbers of probes, from one to millions, hundreds of millions, or billions of probes that may be organized into arrays of other structures. In the following discussion, a single-probe-type sensor is discussed. For example, in the portion of the sensor 202 shown in FIG. 2, all of the probes, such as probe 206, are designed to bind, or lightly associate with, one particular type of target. However, sensors may include many different types of probes, multi-functional probes that bind to, or associate with, multiple types of targets, and various different types of chemical and/or electromechanical probes, and embodiments of the present invention are applicable to all of these different types of sensors.

In other to determine the target concentration in the exemplary solution shown in FIG. 1, the sensor is exposed to a small volume of solution 209, shown above the sensor substrate 208 in FIG. 2. Targets within the solution, such as target 210, randomly collide with, and bind to, probes, such as probe 212. Binding may occur by various different types of binding interactions, including ionic interactions, hydrogen bonding. Van der Waals interactions, covalent bonding, electrostatic surface attractions, and other types of binding. In many cases, binding interactions are quite specific. Targets may bind to probes with binding constants of many orders of magnitude greater than non-targets, including specific, binding of biopolymer probes containing binding sites for specific small-molecule or biopolymer targets. Enzymes bind to small-molecule substrates, antibodies bind to specific antigens, and DNA-binding proteins and RNA biopolymers may bind with high specificity to particular monomer subsequences. However, useful sensors may also include less specific probes that bind less specifically to an entire class of targets.

In certain cases, after an adequate exposure time, the sample solution is washed away from the sensor surface, leaving targets bound to a certain percentage of the probes, including bound targets 214-216 in FIG. 2. In other cases, including sensors used for certain types of impedance spectroscopy, a washing or solution-substitution step is not needed. The percentage of probes bound to targets is generally representative of the concentration of target in the sample solution. Following a washing or solution-substitution step, in certain cases, or during, the binding process, in other cases, the sensor is then queried, by any of various electrical or optical means, in order to detect a signal indicative of the density of bound targets on the sensor substrate, in turn indicative of target concentration in the sample solution. For example, chemiluminescent-compound-labeled biopolymer targets bound to oligonucleotide probes of a microarray can be detected by illuminating the microarray with light of a first wavelength and detecting light of a second, generally longer wavelength emitted by the chemiluminescent labels. In another example, the surface plasmon resonance technique detects biopolymers adsorbed to surfaces by plasmon-induced changes in light reflected from the surface, including changes in reflection angles, wavelengths, and other changes in the reflected light. In microcantilever or nanocantilever transducers, mechanical bending of the nanocantilever as a result of the weight of adsorbed molecules generates a current or voltage signal that varies with the degree to which the cantilever is bent. Atomic-force microscopes move a tiny needle across a surface, positioned at a constant distance from the surface by feedback control, which generates electrical signals as the needle rises and falls as it passes over substrate atoms and adsorbed ions and molecules. As yet another example, fluorescence-resonance-energy-transfer-based detectors may detect the proximity of different fluorescent labels bound to targets and probes by emitting a light signal of a particular wavelength only when targets are bound to or tightly associated with, probes so that the two different fluorophores are within some maximum distance from one another. Finally, in sensors used for impedance spectroscopy, the target-to-probe binding process is detected, while it occurs and with no washing step, by detecting a change in the impedance of an electrode at a frequency at which impedance changes are proportional to bound target density, in turn proportional to target concentration. Impedance spectroscopy takes advantage of the fact that binding of target to probe changes the capacity of the electrode surface.

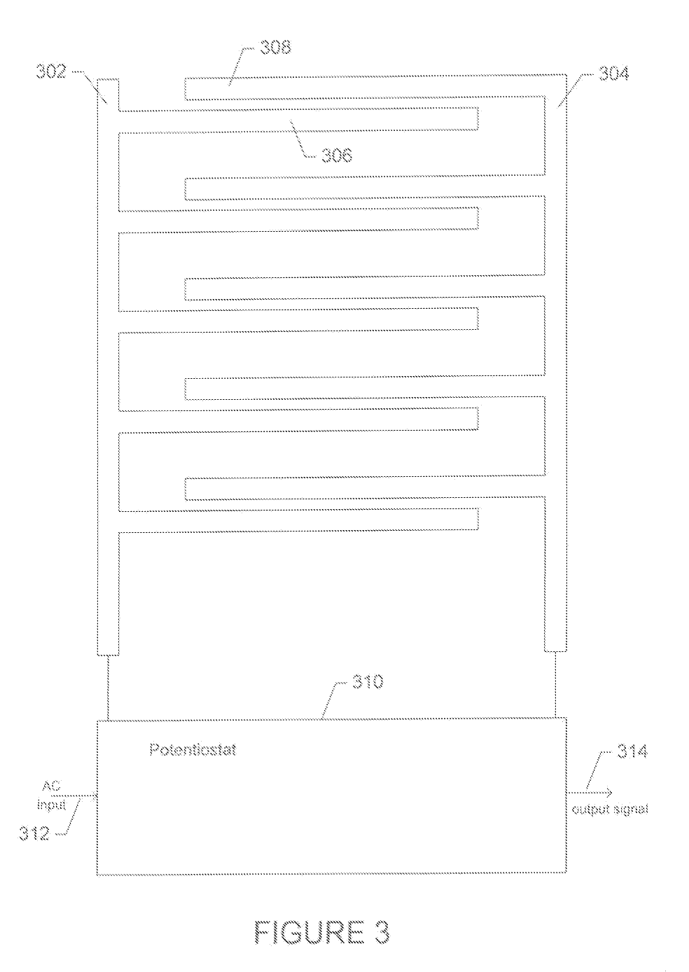

FIG. 3 illustrates an impedance-spectroscopy-based sensor. The sensor includes a first electrode 302 and a second electrode 304, each with multiple projections, including the pair of projections or tines 306 and 308, similar to teeth of a comb, that greatly increase the area of close contact between the first electrode 302 and second electrode 304. In many impedance-spectroscopy-based sensors, the projections may have complex shapes, including invaginations and protrusions, to further increase the area of close contact between the first and second electrodes. The electrodes are electrically coupled to a potentiostat circuit 310 which receives an alternating-current (“AC”) input 312 and produces an output signal 314, the magnitude of which corresponds to the impedance of an electrical circuit within the sensor that includes the first and second electrodes 302 and 304 and a sample solution between the electrodes.

The impedance of a circuit is given by:

$$Z = \frac{V}{I}$$

where V=phasor voltage=Vₘ∠θ_{V}=Vₘ cos(ωt+θ)=R_{c}Vₘe^{j(art+0)}
I=phasor current=Iₘ∠θ_{V}+φ=Iₘ cos(ωt+θ+φ)=R_{c}Iₘe^{j(ν/η+0+φ)}
   The impedance of a circuit depends on the frequency, ω, of the alternating current and alternating voltage within the circuit. Unless the circuit is purely resistive, the voltage and current phases differ by a phase angle φ that represents a combination of phase changes introduced by capacitivc and inductive circuit elements. The impedance is a function of frequency ω and can be expressed as:

Z(jω)=R(ω)+jX(ω)

where R is the real, or resistive component of the impedance and X is the imaginary, or reactive component of the impedance. The impedance is clearly a complex number, and can be written in phasor notation or an exponential notation based on Eider's equation as follows:

Z−Zφθ_{z}=|Z|e^{j}^{ⱼ}²,

As is well known in electronics, the resistance of a direct-current (“DC”) circuit is expressed as:

$$R = \frac{V}{i}$$

where R is the resistance,

V is the voltage, and

i is the current.

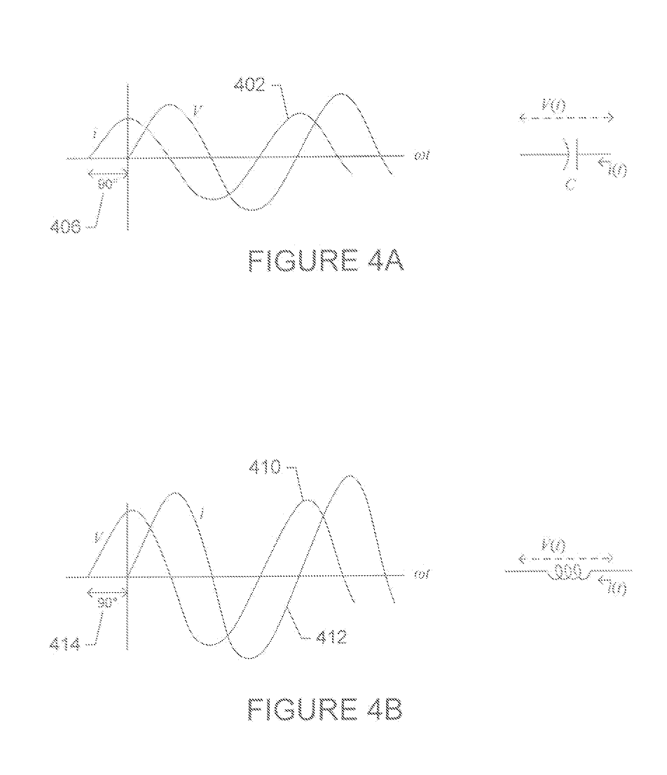

A purely resistive component, or resistor, within either a DC or AC circuit causes a voltage drop across the resistor, and does not induce a phase change between time-domain oscillations, or waveforms, of voltage and current in an AC circuit. By contrast, capacitors and inductors produce phase changes between the voltage and current waveforms in an AC electrical circuit. FIGS. 4A-B illustrate the relative phase changes of current and voltage waveforms in an AC electrical circuit induced by a capacitor and inductor, respectively. As shown in FIG. 4A, the phase of the current waveform 402 leads the phase of the voltage waveform 404 by 90° 406 within a capacitor. As shown in FIG. 4B, the voltage waveform 410 leads the current waveform 412 in phase by 90° 414 within an inductor. The impedance of an AC circuit includes a purely resistive component R, a capacitive reactance component X_{C}, and an inductive reactant component X_{L}:

$$R = \frac{V ∠θ}{I ∠θ} R ∠0°$$

$${X}_{C} = \frac{V ∠θ}{1 ∠θ + 90 °} = \frac{\vert V \vert}{\vert I \vert} ∠ - 90 ° = \frac{1}{ω C} ∠ - 90 °$$

$${X}_{L} = \frac{V ∠θ + 90 °}{1 ∠ θ} = \frac{\vert V \vert}{\vert I \vert} ∠90° = ω L ∠90°$$

The total impedance of an AC circuit is computed from the impedance of each circuit element, and generally includes all three of the purely resistive, capacitive reactance, and inductive reactance components. Impedance is the AC analog of resistance in DC circuits.

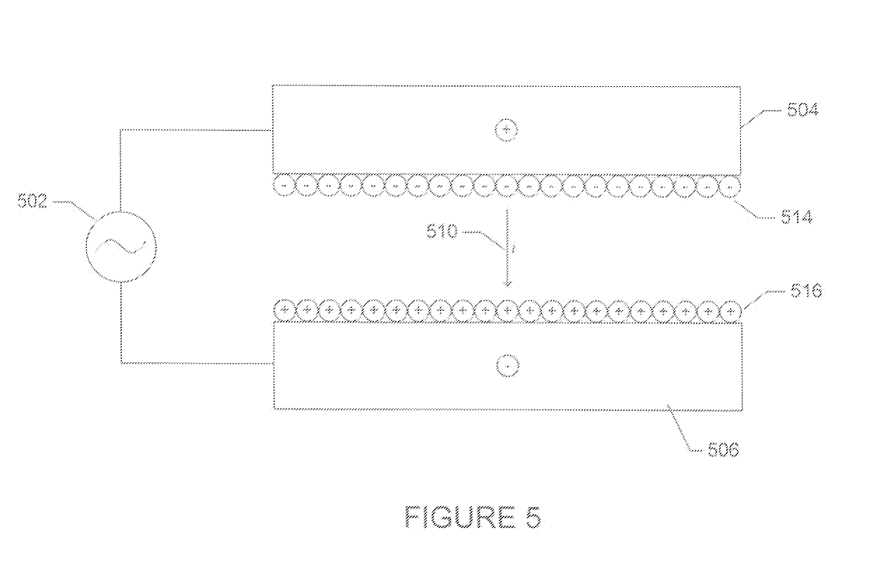

FIG. 5 illustrates a simple form of the impedance-spectroscopy-based sensor discussed above with reference to FIG. 3. The impedance-spectroscopy-based sensor includes an alternating current source 502, a first electrode 504, and a second electrode 506. Current flows between the first electrode 504 and second electrode 506, as indicated by arrow 510 in FIG. 5, through the solution or medium between the two electrodes. The first electrode 504, shown as the positive electrode, or cathode, in FIG. 5, accumulates a layer of negatively charged ions and/or polarizable solutes 514 and the negatively charged electrode 506, or anode, accumulates a layer of positively charged ions and/or polarizable solutes. This accumulation of ions and polarizable solutes on the surface of the electrode, as well as probes bound to, or associated with, the surface of the electrode produces a capacitance.

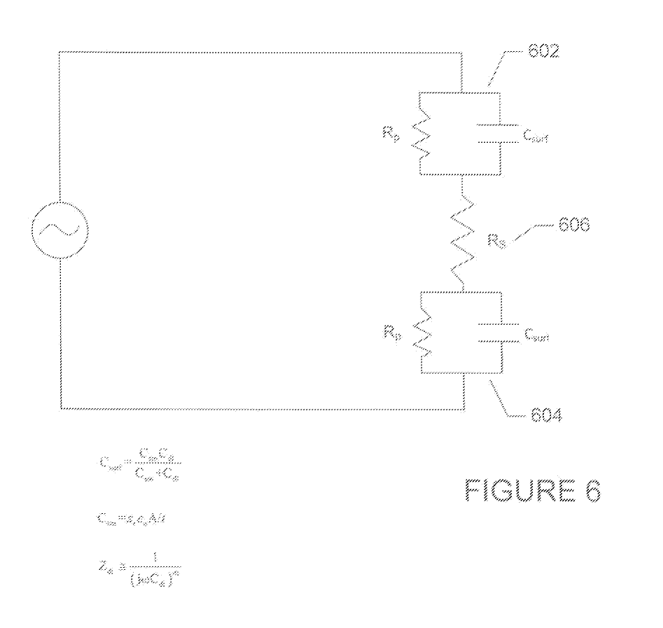

The sensor shown in FIG. 5 can be modeled by an equivalent circuit. FIG. 6 shows the equivalent circuit for the sensor shown in FIG. 5. The first electrode and layer of bound ions and the polarizable solutes is modeled by a resistor, which models polarization-induced resistance, in parallel with a capacitor 602, and the second electrode (506 in FIG. 5) is also modeled by a resistor in parallel with a capacitor 604. The solution path of the current (510 in FIG. 5) is modeled by a solution resistance 606. The capacitor C_{surf} in each capacitor/resistor subcircuit that models each of the first and second electrodes, 602 and 604, may be further modeled as a serial pair of capacitors Cₛₘ and C_{dl} representing the capacitance resulting from modification of the surface of the electrode by covalent attachment or association of probe molecules to the electrode and the capacitance due to a layer of ions and/or polarizable solutes bound to the surface of the electrode:

$${C}_{surf} = \frac{{C}_{sm} {C}_{dl}}{{C}_{sm} + {C}_{dl}}$$

The surface-modification capacitance Cₛₘ is often modeled as:

Cₛₘ=εₜεₒA/t

εₒ is the electrical permittivity;

εₜ is the relative permittivity;

A is the electrode area; and

t is the thickness of the probe layer.

The impedance due to the double-layer capacitance. C_{dl}, may be modeled as:

$${Z}_{dl} ≅ \frac{1}{{( j ω {C}_{dl} )}^{m}}$$

where m ranges from 0.5 to 1.0. In other words, the double-layer capacitance, C_{dl}, introduces a phase change different from 90°.

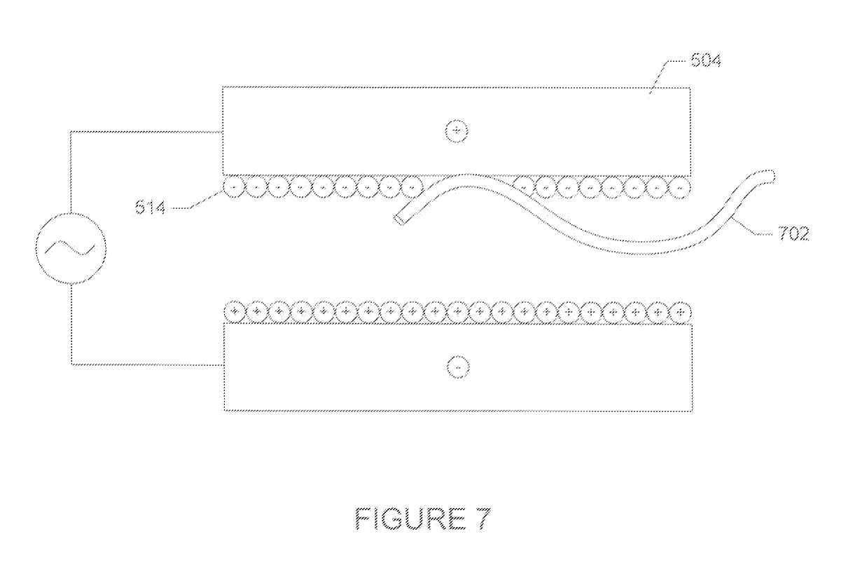

FIG. 7 illustrates the sensor shown in FIG. 5 with a bound target biopolymer. In FIG. 7, the target biopolymer 702 is bound to the first electrode, or cathode 504. Binding of the target biopolymer 702 may displace or reorganize a portion of the layer of ions and polarizable solutes 514, and thus change the value of C_{dl} for the electrode. In addition, binding of the target biopolymer may change the surface-modification capacitance Cₛₘ. In certain cases, the resistance Rₚ, or polarization resistance, shown in FIG. 6 in parallel with the capacitor in each resistor/capacitor model for an electrode, may also be affected by binding the target biopolymer. Many different hypotheses and theories have been proposed for specific physical changes in the electrode surfaces and electrode-surface environments due to target-biopolymer binding to probe molecules on the surface of the sensor, but, in general, definitive physical explanations have so far eluded researchers. However, whatever the exact physical chemical processes involved, binding of target biopolymers to impedance-spectroscopy-based sensors, as shown in FIG. 7, produces detectable changes in the impedance of the electrode circuit, modeled by the equivalent circuit shown in FIG. 6.

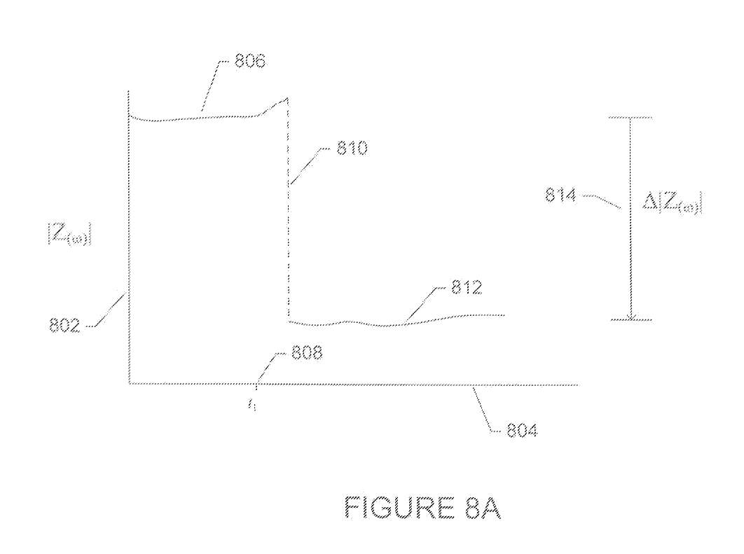

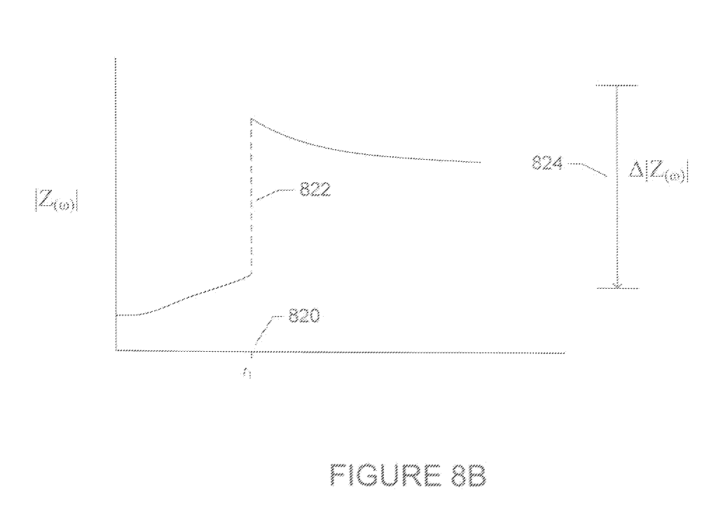

FIGS. 8A-B illustrate impedance-versus-time curves generated during introduction of a biopolymer target into the solution in which an impedance-spectroscopy-based sensor is immersed. Both FIGS. 5A-B use the same illustration conventions, next discussed with reference to FIG. 8A. In FIG. 8A, the magnitude of the impedance |Z(ω)| of the sensor circuit at a particular selected AC frequency, ω, is plotted with respect to the vertical axis 802 and time is plotted with respect to the horizontal axis 804. As shown in FIG. 8A, the initial impedance of the sensor, in the absence of the target biomolecule, is represented by a first portion of the impedance versus time curve 806. After the introduction of the target biopolymer into the solution in which the sensor is immersed, at time t₁ 808, the impedance slightly rises and then falls precipitously, as represented by a dashed line 810, to a relatively smaller impedance value 812. Thus, binding of the target biopolymer results in a decrease in the impedance of the sensor circuit 814. Because impedance is inversely related to the capacitive reactance of the circuit, the drop in impedance may result from an increase in one or both of Cₛₘ and C_{dl} for the electrode to which the target biopolymer binds, a decrease in the polarization resistance Rₚ for the electrode to which the target biopolymer binds, or a combination of a decrease in capacitance and an increase in resistance of the electrode to which the target biopolymer binds, in other cases, as shown in FIG. 8B, introduction of the target biopolymer at time t₁ 820 results in a steep increase, represented by vertical dashed line 822, in the impedance of the sensor circuit. The increase in impedance may result from a decrease in one or both of Cₛₘ and C_{dl} for the electrode to which the target biopolymer binds, an increase in the polarization resistance Rₚ for the electrode to which the target biopolymer binds, or a combination of a decrease in capacitance and an increase in resistance of the electrode to which the target biopolymer binds. In both cases, the magnitude of a change in impedance, upon binding of target to the electrode surface, may be functionally related to the number or density of bound target, in turn proportional to the concentration of the target in a sample solution to which the sensor is exposed.

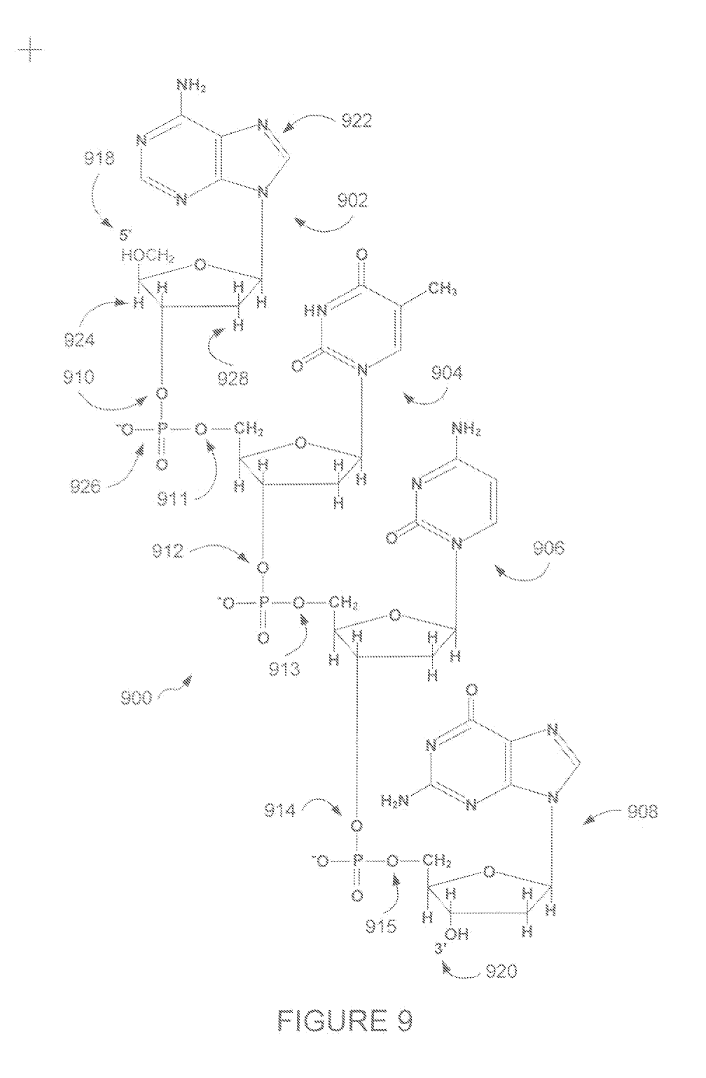

FIG. 9 illustrates a short oligonucleotide. The oligonucleotide is composed of the following subunits: (1) deoxy-adenosine 902; (2) deoxy-thymidine 904; (3) deoxy-cytosine 906; and (4) deoxy-guanosine 908. When phosphorylated, subunits of deoxyribonucleic-acid (“DNA”) and ribonucleic-acid (“RNA”) polymers are called “nucleotides” and are linked together through phosphodiester bonds 910-915 to form DNA and RNA polymers. A linear DNA molecule, such as the oligomer shown in FIG. 9, has a 5′ end 918 and a 3′ end 920. A DNA polymer can be chemically characterized by writing, in sequence from the 5′ end to the 3′ end, the single letter abbreviations for the nucleotide subunits that together compose the DNA polymer. For example, the oligomer 900 shown in FIG. 9 can be chemically represented as “ATCG.” A DNA nucleotide comprises a purine or pyrimidine base (e.g. adenine 922 of the deoxy-adenylate nucleotide 902), a deoxy-ribose sugar (e.g. deoxy-ribose 924 of the deoxy-adenylate nucleotide 902), and a phosphate group (e.g. phosphate 926) that links one nucleotide to another nucleotide in the DNA polymer.

The DNA polymers that contain the organization information for living organisms occur in the nuclei of cells in pairs, forming double-stranded DNA helixes. One polymer of the pair is laid out in a 5′ to 3′ direction, and the other polymer of the pair is laid out in a 3′ to 5′ direction. The two DNA polymers in a double-stranded DNA helix are therefore described as being anti-parallel. The two DNA polymers, or strands, within a double-stranded DNA helix are bound to each other through attractive forces including hydrophobic interactions between stacked purine and pyrimidine bases and hydrogen bonding between purine and pyrimidine bases, the attractive forces emphasized by conformational constraints of DNA polymers. Because of a number of chemical and topographic constraints, double-stranded DNA helices are most stable when deoxy-adenylate subunits of one strand hydrogen bond to deoxy-thymidylate subunits of the other strand, and deoxy-guanylate subunits of one strand hydrogen bond to corresponding deoxy-cytidilate subunits of the other strand. The two strands have complementary sequences.

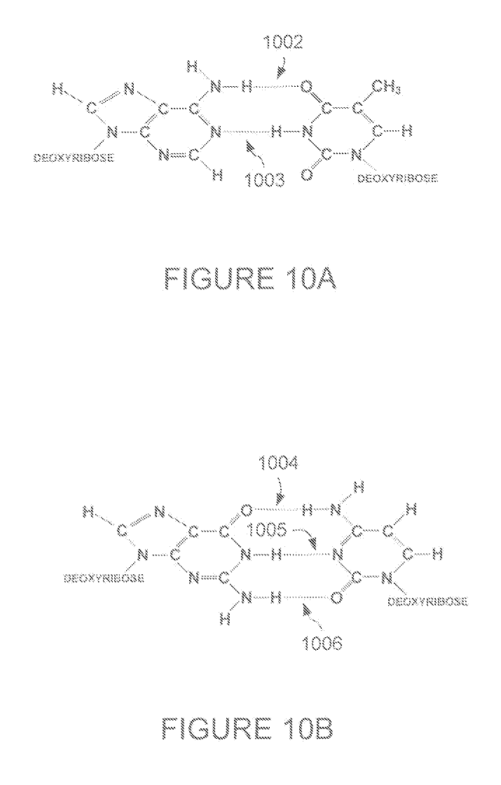

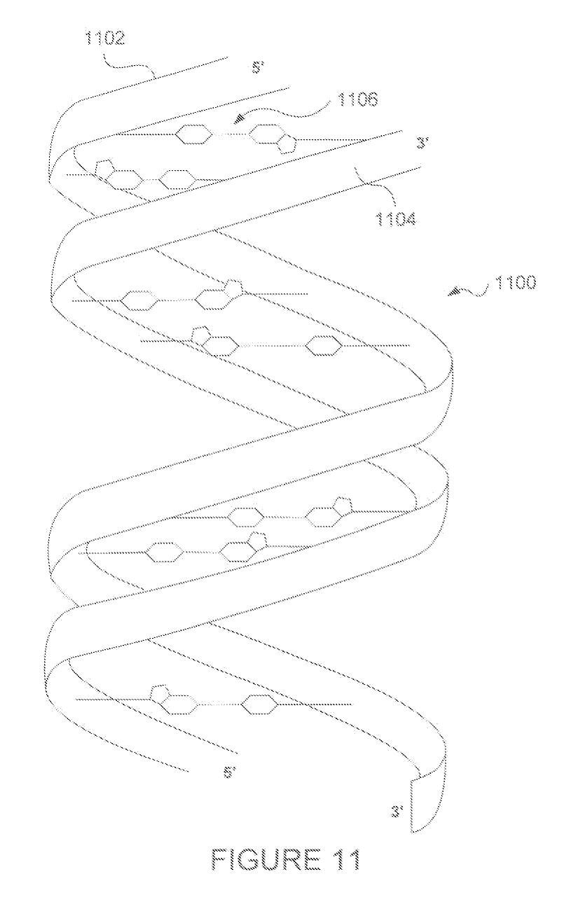

FIGS. 10A-B illustrate the hydrogen bonding between the purine and pyrimidine bases of two anti-parallel DNA strands. AT and GC base pairs, illustrated in FIGS. 10A-B, are known as Watson-Crick (“WC”) base pairs. Two DNA strands linked together by hydrogen bonds forms the familiar helix structure of a double-stranded DNA helix. FIG. 11 illustrates a short section of a DNA double helix comprising a first strand and a second, anti-parallel strand.

In the following examples of embodiments of the present invention, an impedance-spectroscopy-based sensor is used to detect the presence of single-stranded DNA or RNA in a solution and to quantify the concentration of the target single-stranded DNA or RNA biopolymer, which is proportional to a change in impedance of the sensor, as discussed above with reference to FIGS. 8A-B, when the target single-stranded DNA or RNA biopolymer binds to probes bound to an electrode surface. The probe molecules, in these examples, are oligonucleotides complementary to a subsequence of the target single-stranded RNA or DNA and are covalently bound to the electrode surface by any of various functionalization techniques. For example, a thiol-functionalized alcohol can be used to bind the oligonucleotide to a gold electrode, with the sulfur atom binding to the cold electrode and the oxygen atom of the OH group of the alcohol replacing an oxygen atom of a phosphodiester phosphorous atom at either the 3′ or 5′ end of the oligonucleotide.

The oligonucleotide probes may be varied in length and sequence to provide complementary binding to complementary subsequences of the target biomolecule of a desired thermodynamic stability. In general, the strength of binding the target biomolecule to a complementary probe oligonucleotide is proportional to the length of the complementary oligonucleotide. In general, the binding needs to be sufficiently strong to produce a strong impedance-chain signal for sensitive detection of the biopolymer target, but should not be too strong to interfere with subsequent removal of the biopolymer target and reuse of the sensor to determine the presence and concentration of the target biopolymer in subsequent sample solutions.

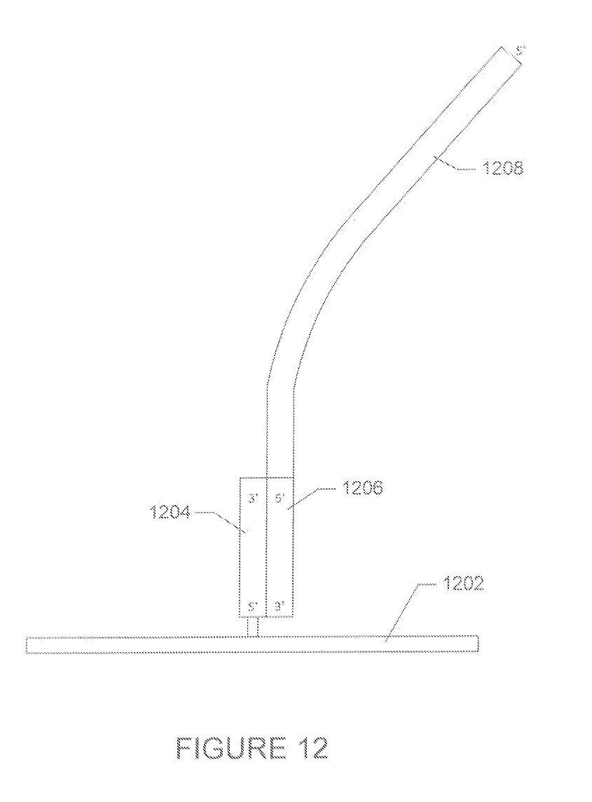

FIG. 12 illustrates an approach generally used for binding target molecules to impedance-spectroscopy-sensor electrodes. The electrode surface 1202 is generally functionalized to contain a single probe oligonucleotide 1204 complementary to a single binding site 1206 within the target biopolymer 1208. In certain applications, the sensor electrode may be functionalized to contain two or more types of oligonucleotide probes, but in currently available technologies, each of the different types of probe is designed to bind to a different target biopolymer, and each target biopolymer binds to a single probe. This technology provides very specific binding of target biopolymers to sensors, and has been used for a variety of extremely sensitive and useful analysis and diagnostic techniques. However, in many cases, particularly when the concentration of target biopolymer in sample solutions is extremely low, the change in sensor impedance, in the case of impedance-spectroscopy-based sensors, may be of insufficient magnitude to generate signal-to-noise ratios adequate for reliable and accurate quantification of extremely low concentration of target biopolymers in sample solutions. Even in the case of more concentrated sample solutions, the signal-to-noise ratios may be nonetheless inadequate, due to various different modes of non-specific binding or association of non-target biopolymers and solutes to the electrode surfaces.

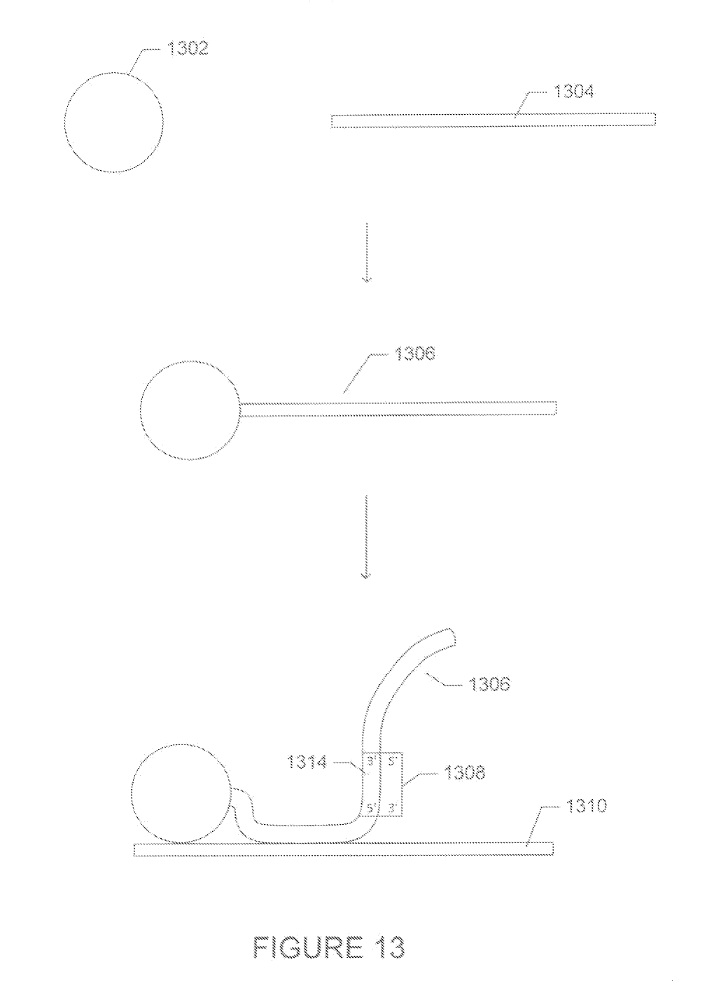

FIG. 13 illustrates the approach of certain embodiments of the present invention to enhance the signal strength and signal-to-noise ratio of impedance-spectroscopy-based sensors. As shown in FIG. 13, in various embodiments of the present invention, a nanoparticle 1302 is combined with a target 1304, in a sample solution or another medium, to produce a nanoparticle/target complex 1306. The nanoparticle/target complex 1306 is then specifically bound to an oligonucleotide probe 1308 associated with, or covalently bound to, the substrate 1310 of an impedance-spectroscopy-based biosensor electrode. The oligonucleotide probe 1308 has a sequence complementary to a subsequence within the target. Presence of the nanoparticle on or near the electrode surface further alters the capacitance, modeled as C_{surf}, as discussed above, that is modified by the bound target to produce a stronger impedance-change signal and larger signal-to-noise ratio. As one example, the nanoparticle may have a relative permittivity, εₜ, significantly different from that of the probe oligonucleotides, and thus result in a significant change in the surface-modification capacitance, Cₛₘ, for the electrode to which the nanoparticle/target complex binds. The nanoparticle may also alter the polarization resistance Rₚ, and/or alter the organization and characteristics of the ion/polarizable solute layer associated with the electrode. Because the nanoparticle may be composed of very different types of materials and compounds than the target molecules and other solutes, the nanoparticle may have significantly greater effect on the capacitance and polarization resistance of a sensor electrode than target molecules or other sample-solution solutes. Nanoparticles generally have diameters of from between 1 and 10 nanometers, but may have diameters of from 0.5 to 50 nanometers, from 1 to 100 nanometers, or from 1 nanometer up to, but less than, 1 micrometer.

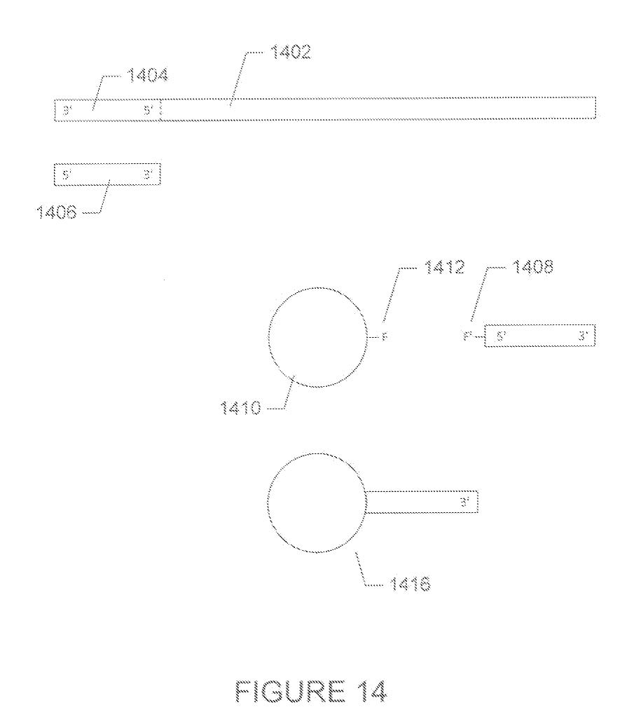

There are a number of different approaches to generating a nanoparticle/target complex from a target-containing sample solution. FIGS. 14-15 illustrate a target-amplification method for generating a nanoparticle/target complex according to one embodiment of the present invention. The target molecule 1402 can be considered to include a leading 3′ subsequence 1404. A complementary oligonucleotide 1406 to the leading 3′ subsequence 1404 of the target molecule is synthesized, by any of routine oligonucleotide synthesis methods, and functionalized with an active group F′ 1408. Nanoparticles 1410 are functionalized with an active functional group F 1412. The combination of the functionalized oligonucleotide and functionalized nanoparticle, under appropriate solution conditions, including functionalized oligonucleotide and functionalized nanoparticle concentrations, temperature, and other conditions, results in covalent bonding or another type of strong binding of the oligonucleotide primer and nanoparticle to produce a nanoparticle/primer complex 1416.

A number of different chemistries are available for binding the nanoparticle to the primer. For example, F′ may be a primary amine, introduced by modifying the 5′ nucleotide base or derivatizing the 5′ OH group with any of various functionalized aliphatic amines, and the F may be an aldehyde, with binding of the nanoparticle to the primer obtained through formation of a Schiff base by reaction of the primary amine and the aldehyde. Alternatively, F may be a carboxyl group and F′ a primary amine, with binding of the nanoparticle to the primer obtained through formation of an amide by any of various methods, including a carbodiimide intermediate. As yet another alternative, F may be cyanuric chloride and F′ a primary amine, with binding of the nanoparticle to the primer obtained through substitution of the primary amine for one of the chlorine atoms of the cyanuric chloride.

As shown in FIG. 15, the nanoparticle/primer complex 1416 is then introduced into a sample solution containing target molecules 1402, resulting in binding of the nanoparticle/primer complex 1416 to the leading, complementary subsequence 1404 of the target molecules 1402. Introduction of a thermally stable DNA polymerase 1418, such as the Taq polymerase, and nucleotide triphosphates results in a polymerase-chain-reaction (“PCR”) synthesis of a complementary nanoparticle/target complex 1420 (“cNTcomplex”) bound to the target, which can be dissociated from the target molecule, by heating the solution, to produce a free cNTcomplex 1422. The dissociated target 1424 and then serve as a template tor another cNTcomplex synthesis, and the PCR synthesis of cNTcomplex can be carried out for a fixed number of cycles to generate a cNTcomplex solution with the concentration of cNTcomplex proportional to the initial concentration of target in the sample solution. An impedance-spectroscopy-based sensor electrode with, probe oligonucleotides complementary to a subsequence of the cNTcomplex can then be exposed to the cNTcomplex solution to generate an impedance-change signal from which the initial target concentration can be computed.

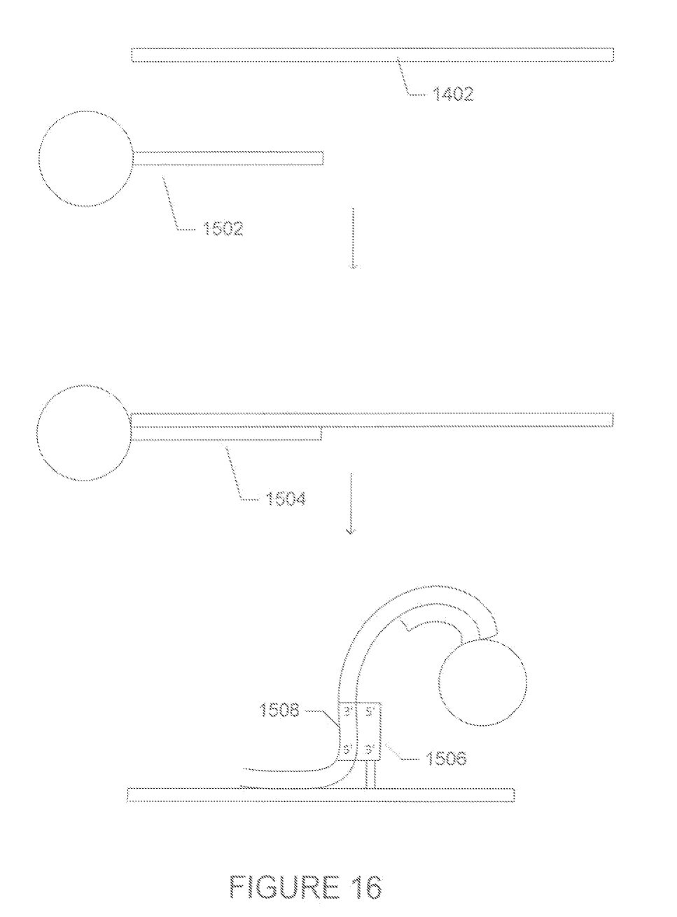

FIG. 16 illustrates an alternative approach to producing nanoparticle-bound target complexes. As shown in FIG. 16, the target 1402 may directly bind with a complementary nanoparticle/oligonucleotide complex 150, synthesized by any of the above-described methods for synthesizing the nanoparticle/primer complex, to produce a nanoparticle-oligonucleotide-complex/target complex 1504 that can then bind to a probe oligonucleotide 1506 on an electrode surface complementary to a subsequence 1508 within the target to generate an impedance-change signal from which the initial target concentration can be computed.

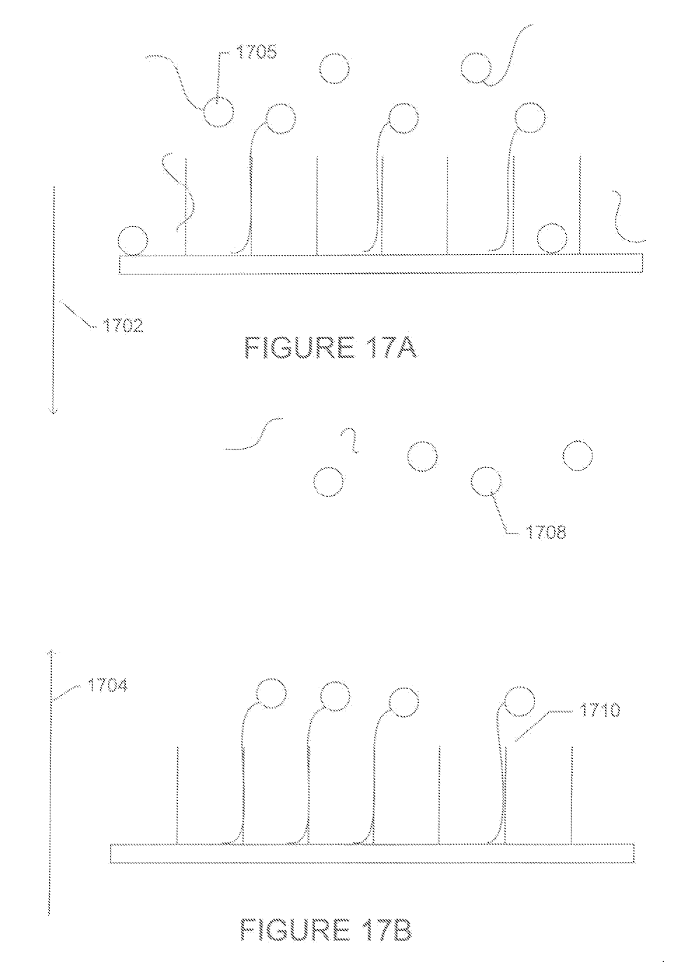

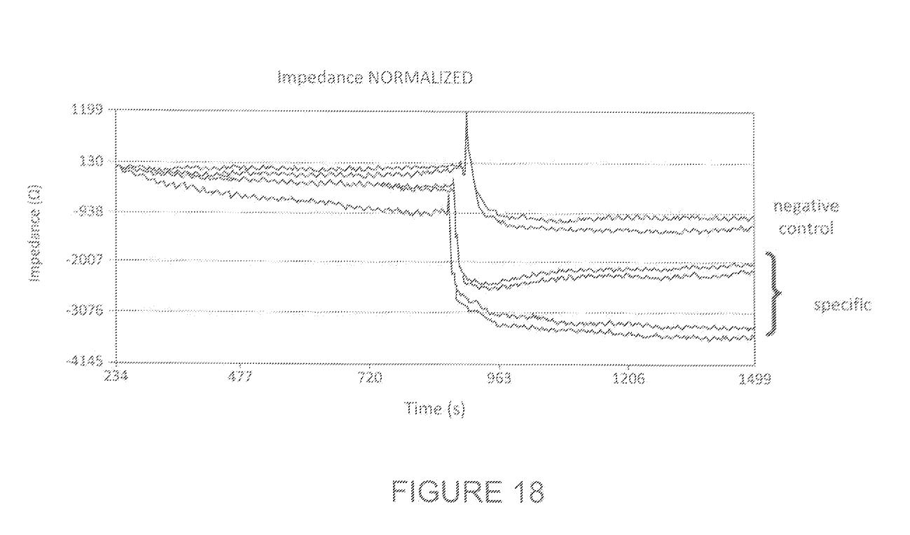

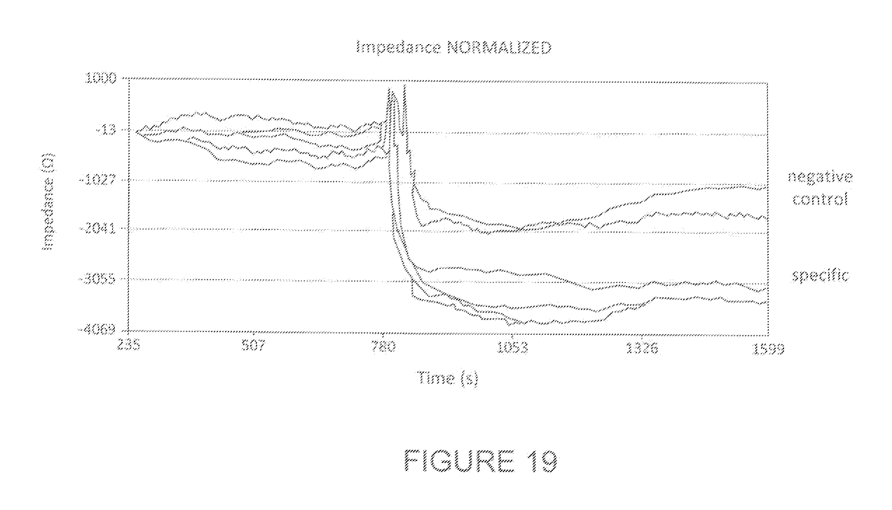

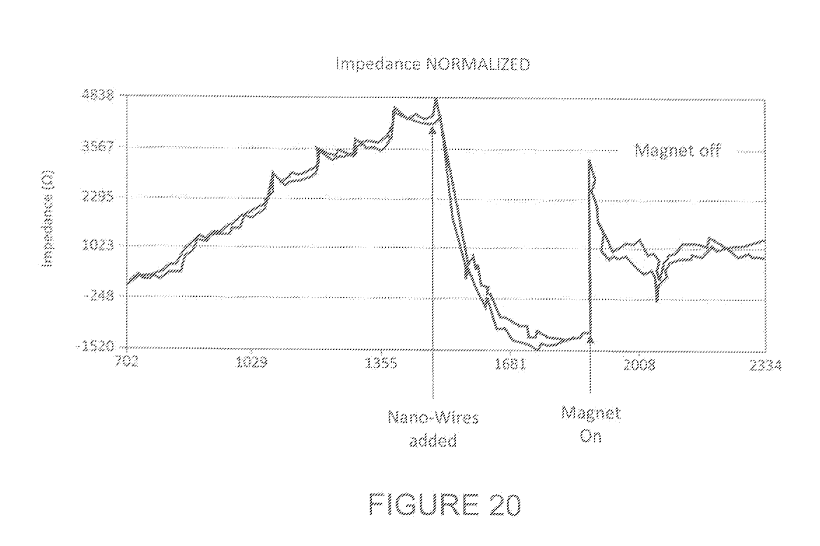

FIGS. 17A-B illustrate an additional feature of nanoparticle/target complexes useful in improving signal-to-noise ratios of impedance signals generated by impedance-spectroscopy-based sensors according to certain embodiments of the present invention. The nanoparticles used to form nanoparticle/target complexes, by any of the above-discussed methods, may be paramagnetic or ferromagnetic, and thus responsive to applied magnetic fields. During exposure of an impedance-spectroscopy-based-sensor electrode to a solution of nanoparticle/target complexes, as shown in FIG. 17A, a magnetic field 1702 is first applied, to drive nanoparticle/target complexes, such as nanoparticle/target complex 1705, toward the electrode surface to increase the concentration of the nanoparticle/target complexes near the surface and promote binding, and then, as shown in FIG. 17B, the field 1704 is reversed, to remove unbound nanoparticles, such as free nanoparticle 1708, from the surface of the electrode, so that the impedance change is nearly entirely due to the presence of nanoparticles within nanoparticle/target complexes bound to oligonucleotide probes 1710. The initially applied magnetic field 1702 may also allow for a washing or fluid-flow step to remove nonspecifically associated solutes and non-target biopolymers from the electrode surface, since the nanoparticle/target complexes are held to the electrode surface both by magnetic forces as well as by complementary binding to oligonucleotide probes.

Many different types of nanoparticles can be used to enhance target molecules and to, in turn, improve the signal strength and signal-to-noise ratio obtained from an impedance-spectroscopy-based sensor. While beads and other spherical and nearly spherical particles are shown, in the figures referenced in the descriptions of embodiments of the present invention, provided above, less symmetrical nanoparticles, including nanorods and nanowires, can also be used, and may provide even greater impedance changes upon binding to sensor probes, for example, conductive nanorods and nanowires may significantly decrease the surface impedance.

### EXPERIMENTAL RESULTS

The experiments, described below, were conducted in furtherance of developing specific nanoparticle-enhanced sample solutions and methods for enhancing impedance signals produced by impedance-spectroscopy-based sensors with oligonucleotide probes. These experiments are intended to illustrate particular methods that represent embodiments of the present invention, but are not intended to, in any way, limit the claims which follow.

### Experiment 1

Aldehyde-terminated magnetic beads having diameters of approximately 1 um, obtained from Chemicell GmbH (Berlin, Germany), were functionalized with the following oligonucleotide probes (1) asP2P5; and (2) asADK. The sequences of these two oligonucleotide probes are provided below:

| | |
|---|---|
| (1) | “AAACAACTAGCAATGGCATTTCGCACTACC;” |
| and | |
| (2) | “ATACCATATTTCTCCATGATGAACTGAGCCTGAGT.” |

Both oligonucleotide probes have amino groups at their 5′ ends. A suspension of the magnetic beads (10 uL of 50 mg/mL) was added to 200 uL of 0.1 M MES buffer, pH 6.1, magnetically separated, and another 200 uL of MES buffer was added. The oligonucleotide probes, (1 μL of 1 mM concentration) was then added to the suspension and incubated for 2 h at ambient temperature. A saturated water solution of glycine (10 uL) was then added and incubated for another 15 min. The beads were then washed three times with SSPE buffer that contained 0.05% of Tween 20 (SSPET) by magnetic separation followed by addition of fresh SSPET. The final suspension contained oligonucleotide functionalized beads in 200 uL of SSPET. Final magnetic-bead concentration in the suspension was 2.5 mg/mL. Impedance arrays were functionalized with either P2P5 probes or ADK probes that were complimentary toward asP2P5 and asADK oligonucleotides correspondingly.

Impedance measurements were performed at 150 mV and 150 Hz. The suspension of functionalized beads was injected to the fluidic cell of the impedance spectroscopy chip. A suspension of magnetic beads functionalized with an oligonucleotide that is not complimentary to the probe on the chip was used as a negative control. Thus, beads functionalized with asP2P5 were used as a negative control for ADK functionalized chip and beads functionalized with asADK was used as a negative control for P2P5 functionalized chip. FIG. 18 shows the impedance response of an ADK-functionalized chip to asADK functionalized beads (specific response) and to asP2P5-functionalized beads (negative control).

Clearly, the impedance decrease caused by injection of the magnetic beads functionalized with the probe-specific oligonucleotide asADK demonstrated a stronger response than magnetic beads functionalized with the negative-control oligonucleotide asP2P5. FIG. 19 shows the impedance response of P2P5 functionalized chip to asP2P5-functionalized beads (specific response) and to asADK-functionalized beads (negative control). Again, the specific response is higher than the response for the negative control. These results show that

functionalized beads bind specifically to a surface of a functionalized chip; and (2) the impedance response obtained as a result of the beads to surface binding can be measured by impedimetric detection.

### Experiment 2

Cyanuric-chloride (“CC”)-modified magnetic beads having diameters of approximately 500 nm, obtained from Chemicell GmbH (Berlin, Germany) were suspended in a buffer solution. The suspension (10 ul of 50 ug/ml) was washed with 0.1 M MES buffer (pH 6.1). The magnetic beads were then re-suspended in 1 ml of the same buffer containing 1 mM of NH2-modified HlyA-F16 forward primer (NH2-CAGTCCTCATTACCCAGCAAC) and incubated for 1 h at room temperature. Glycine solution (20 ul of saturated solution in water) was then added to the suspension and incubated for 30 min. The beads were then washed by multiple stages of magnetic separation and buffer change. Finally, the beads were resuspended in 100 ul of MES buffer and stored at 4° C. The final concentration of the beads suspension was 5 ug/ml.

For PCR amplification, a HlyA-F16 DNA template was used, the template having the sequence:

CAGTCCTCATTACCCAGCAACATTGGGATACGCTGATAGGTGAGTTA
GCTGGTGTCACCAGAAATGGAGACAAAACACTCAGTGGTAAAAGTTA
TATTGACTATTATGAAGAAGGAAAACGTCTGGAGAAAAAACCGGATG
AATTCCAGAAGCAAGTCTTTGACCCATTGAAAGGAAATATTGACCTT
TCTGACAGCAAATCTTCTACGTTATTGAAATTTGTTACGCCATTGTT
AACTCCCGGTGAGGAAATTCGTGAAAGGAGGCAGTCCGGAAAATATG
AATATATTACCGAGTTATTAGTCAAGGGTGTTGATAAATGGACGGTG
AAGGGGGTTCAGGACAAGGGGTCTGT.

Initially the PGR mixture contained the template and both primers. First, 12 thermo-cycles of the PCR were carried out before adding the beads to the mixture. Then the primer modified beads were added to the mixture to form 0.5 ug/mL beads concentration and 30 cycles of PCR were performed additionally. Beads were then separated magnetically from the suspension and supernatant solution was examined by gel electrophoresis. DI water (1.0 ul) was then added to the beads, and the beads suspension was heated at 95 C for 3 minutes to melt double-helix DNA attached to the bead surface. The supernatant was then removed and the following samples tested electrophoretically:

Sample 1 HlyA-F16 PCR control (no beads added)
Sample 2 HlyA-F16 PCR 12 cycles
Sample 3 Hly PCR 0.5 ul/ml beads added after 12 cycles (supernatant obtained after additional 30 cycles)
Sample 4 Hly PCR 0.5 ul/ml beads added after 12 cycles. Supernatant removed after additional 30 cycles, 10 ul DI water added to the beads and beads heated to 95 C then supernatant removed and tested

Electrophoresis results for Sample 4 indicate that Sample 4 contains an extended antisense DNA strand complexed with an extended forward primer attached to bead surface. Thus, the expected extension of the primer attached to bead surface did occur.

### Experiment 3

Nanowires having an approximate length of 17 um and an approximate width of 500 nm were manufactured by using etched Si nanowires on a silicon-on-insulator (“SOI”) wafer as the template for formation of the magnetic nanowires. The Si nanowires are formed by patterning and etching the top Si layer of the SOI wafer and removing the buried oxide from beneath the Si nanowires with an isotropic vapor HF etch. Magnetic material is evaporated onto the Si nanowires using an e-beam evaporator, followed by e-beam evaporation of the gold functionalization material. A lift-off approach that incorporates negative-tone photoresist is used to remove the metal layers from the ends of the nanowires to allow a Si etch to be employed for release of the nanowires into a suspension. In one experiment, an SOI substrate with patterned and etched Si nanowires that have had the buried oxide removed were coated with the magnetic (e.g. cobalt) and functionalizing (e.g. gold) layers. The cobalt and gold structures were released from the SOI wafer with an isotropic etch that removed the regions of Si that pin the ends of the etched Si nanowires, as well as the Si nanowire itself.

The Co layer provided magnetic properties and the Au surface layer provided functionalization ability and conductivity. The nanowires were not functionalized. Sedimentation of the rods over the die surface was carried out by gravity. A non-functionalized impedance spectroscopy chip with 10 um gap between electrodes was used. The sedimentation of the nanowires is intended to decrease impedance due to ‘shortening’ the gap between two electrodes of impedance spectroscopy electrode pair.

A suspension of nanowires in buffer solution for introduction into the fluidic chamber of the impedance chip was performed. FIG. 20 shows that addition of a nanowire suspension results in a rapid decrease in impedance of an impedance-spectroscopy-based sensor due to sedimentation of the conductive rods over the chip surface. Application of a magnetic field from the above of the cell for a short time increases the impedance almost back to the initial level. Switching the magnetic field off results in sedimentation of nanowires back onto the chip surface and, as a result, a decrease in array impedance is again observed.

Although the present invention has been described in terms of particular embodiments, it is not intended that the invention be limited to these embodiments. Modifications will be apparent to those skilled in the art. For example, the sequences and lengths oligonucleotide probes vary for different target molecules and applications. In general, in order to provide sufficiently strong binding to produce a strong impedance-change signal and large signal-to-noise ratio, but to prevent irreversible binding of target biopolymers to the functionalized electrode surface, oligonucleotide-probe sequences are chosen to provide binding constants that allow for strong, sequence-specific binding of target molecules but that also allow for target molecules to be removed by relatively mild changes in solution conditions, including temperature and/or ionic strength. Nanoparticles can be made from metals, metal oxides, organic compounds, ceramic materials, and various nanofabricated material by well-known processes. Nanoparticles useful for functionalizing targets for sensing by impedance-spectroscopy-based sensors include those nanoparticles that generate significant capacitance changes when bound to the electrode surface.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit die invention to the precise forms disclosed. Many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A sample solution comprising:
non-target solutes; and
a nanoparticle/target-biopolymer complex, a nanoparticle component of the nanoparticle/target-biopolymer complex producing a change in the capacitance of an impedance-spectroscopy-based-sensor electrode when bound to, or near to, a surface of an impedance-spectroscopy-based-sensor electrode and a target-biopolymer component of the nanoparticle/target-biopolymer complex specifically binding to probes associated with, or bound to the impedance-spectroscopy-based-sensor electrode.

2. The sample solution of claim 1 wherein the nanoparticle component of the nanoparticle/target-biopolymer complex is a roughly spherical particle of diameter between 1 and 1000 nanometers and wherein the nanoparticle component comprises one or more of:
a metal;
a metal oxide;
a semiconductor;
a grapheme-like carbon network;
an organic compound;
an organic polymer; and
a ceramic material.

3. The sample solution of claim 1 wherein the nanoparticle component of the nanoparticle/target-biopolymer complex is a rod-like nanowire or nanorod particle having a width of between 1 and 1000 nanometers and wherein the nanoparticle component comprises one or more of:
a metal;
a metal oxide;
a semiconductor;
a grapheme-like carbon network;
an organic compound;
an organic polymer; and
a ceramic material.

4. The sample solution of claim 1 wherein the target-biopolymer component of the nanoparticle/target-biopolymer complex is one of:
a ribonucleic-acid polymer; and
a deoxyribonucleic polymer.

5. A sensor comprising:
a substrate;
a signal-generation component coupled to the substrate that produces a sensor signal;
probes associated with, or bound to, the substrate, each probe binding to binding site of a target, so that, when the sensor is exposed to the target, the target is bound to a probe to produce a change, in one or more physical characteristics of the substrate, probes, and/or other substrate-associated entities, that is detected by the signal-generation component, which generates a corresponding sensor signal; and
a nanoparticle/target-biopolymer complex, including a nanoparticle component and a target-biopolymer component, bound to one or more probes.

6. The sensor of claim 5 wherein the nanoparticle component of the nanoparticle/target-biopolymer complex is a roughly spherical particle of diameter between 1 and 1000 nanometers and wherein the nanoparticle component comprises one or more of:
a metal;
a metal oxide;
a semiconductor;
a grapheme-like carbon network;
an organic compound;
an organic polymer; and
a ceramic material.

7. The sensor of claim 5 wherein the nanoparticle component of the nanoparticle/target-biopolymer complex is a rod-like nanowire or nanorod particle having a width of between 1 and 1000 nanometers and wherein the nanoparticle component comprises one or more of:
a metal;
a metal oxide;
a semiconductor;
a grapheme-like carbon network;
an organic compound;
an organic polymer; and
a ceramic material.

8. The sensor of claim 1 wherein the target-biopolymer component of the nanoparticle/target-biopolymer complex is one of:
a ribonucleic-acid polymer; and
a deoxyribonucleic polymer.

9. A method for detecting and/or quantifying an amount of a target molecule in a sample solution, the method comprising:
derivatizing the target to produce a nanoparticle/target complex, including a nanoparticle component and a target-biopolymer component, in the sample solution;
applying the sample solution to an impedance-spectroscopy-based sensor; and
detecting and/or quantifying an initial amount of the target molecule in the sample solution by detecting a change in a signal output by the impedance-spectroscopy-based sensor.

10. The method of claim 9 wherein the nanoparticle component of the nanoparticle/target-biopolymer complex is a roughly spherical particle of diameter between 1 and 1000 nanometers and wherein the nanoparticle component comprises one or more of:
a metal;
a metal oxide;
a semiconductor;
a grapheme-like carbon network;
an organic compound;
an organic polymer; and
a ceramic material.

11. The method of claim 9 wherein the nanoparticle component of the nanoparticle/target-biopolymer complex is a rod-like nanowire or nanorod particle having a width of between 1 and 1000 nanometers and wherein the nanoparticle component comprises one or more of:
a metal;
a metal oxide;
a semiconductor;
a grapheme-like carbon network;
an organic compound;
an organic polymer; and
a ceramic material.

12. The method of claim 9 wherein the target-biopolymer component of the nanoparticle/target-biopolymer complex is one of:
a ribonucleic-acid polymer; and
a deoxyribonucleic polymer.

13. The method of claim 12 wherein derivatizing the target to produce a nanoparticle/target complex, including a nanoparticle component and a target-biopolymer component, in the sample solution further includes:
combining the nanoparticle with a functionalized primer oligonucleotide complementary to a subsequence of the target to produce a nanoparticle/primer complex;
synthesizing a complementary nanoparticle/target complex by a polymerase chain reaction; and
melting the target from the complementary nanoparticle/target complex.

14. The method of claim 12 wherein derivatizing the target to produce a nanoparticle/target complex, including a nanoparticle component and a target-biopolymer component, in the sample solution further includes:
combining the nanoparticle with a functionalized tag oligonucleotide complementary to a subsequence of the target to produce a nanoparticle/tag complex; and
binding the nanoparticle/tag complex to the target.
